(19) United States
(12) Patent Application Publication
(10) Pub. No.: US 2026/0137266 A1
(43) Pub. Date: May 21, 2026

(54) ENDOSCOPE ADAPTOR SYSTEM AND METHOD

(71) Applicant: MARZ Surgical, LLC, Seattle, WA (US)

(72) Inventors: Kris Moe, Seattle, WA (US); Jeremy Ruthberg, Seattle, WA (US); Lingga Adidharma, Seattle, WA (US); Peiran Zhou, Seattle, WA (US)

(21) Appl. No.: 19/397,069

(22) Filed: Nov. 21, 2025

Related U.S. Application Data
(60) Provisional application No. 63/723,314, filed on Nov. 21, 2024.

(51) Int. Cl. A61B 1/00; A61B 1/005

(52) U.S. Cl. CPC A61B 1/0014; A61B 1/00042; A61B 1/00128; A61B 1/0052

(57) ABSTRACT

A method of using an endoscope that includes performing an endoscopy task with an endoscope. The endoscope includes a rod that is coupled to the endoscope and that extends from a rod base to a rod end. Performing the endoscopy task with the endoscope includes manipulating, by a hand of a user, a neck of the endoscope to move the neck along the length of the rod and to extend and retract a head of the neck a desired length past the rod end to perform the endoscopy task while the user is holding the endoscope.

### CROSS-REFERENCE TO RELATED APPLICATIONS

This application is a non-provisional of and claims the benefit of U.S. Provisional Application No. 63/723,314, filed November 21, 2024, entitled “FLEXIBLE RIGID ENDOSCOPE ADAPTOR (FRESA) SYSTEM AND METHOD,” with attorney docket number 0124089-001PR0. This application is hereby incorporated herein by reference in its entirety and for all purposes.

### BACKGROUND

Endoscopy is an expanding market both nationally and globally, with two primary types of endoscopes typically used to visualize the sinonasal cavity: flexible and rigid endoscopes. Flexible endoscopes are generally more affordable, widely accessible, and can require two-handed operation, as a button or trigger can be used to change the direction of the tip in various examples. Additionally, flexible endoscopes can be used to look beyond the nasal cavity into the larynx. On the other hand, rigid endoscopes are static, and can allow for one-handed use, which frees the other hand for additional instrumentation. As a result, rigid endoscopes are often preferred for surgical sinonasal procedures, while flexible endoscopes are more commonly used for diagnostic and visualization purposes.

Rigid endoscopy often requires a significant amount of additional equipment, including sterile scopes, light sources, camera systems, video towers, and specialized staff training, which may not always be readily available in clinical settings. In some cases, operators may initially opt for a flexible endoscope to reduce costs and allow better visualization of multiple areas, only to find that a rigid endoscope is needed to perform a procedure following diagnosis (e.g., biopsy, etc.). Currently, no solution exists that allows for the seamless conversion of a given flexible endoscope into a rigid one or integrates the clinical utility of a one-handed flexible endoscope. This gap highlights the need for a device that offers both flexibility and enhanced functionality in clinical environments.

In view of the foregoing, a need exists for an improved flexible to rigid conversion system and method for accomplishing flexible to rigid endoscopic conversion in an effort to overcome the aforementioned obstacles and deficiencies of conventional endoscopic systems.

### BRIEF DESCRIPTION OF THE DRAWINGS

FIG. 1a is an exemplary perspective view illustrating an embodiment of an endoscope adaptor system in accordance with an embodiment.

FIG. 1b is an exemplary side view illustrating the endoscope adaptor system of FIG. 1a.

FIG. 1c is an exemplary second perspective view illustrating the endoscope adaptor system of FIGS. 1a and 1b.

FIG. 2 is an exemplary side view illustrating a flexible endoscope neck coupled to an endoscope adaptor system with the head of the endoscope extending past the distal end of the rod in accordance with an embodiment.

FIG. 3 is an exemplary side view illustrating a user holding an endoscope with an endoscope adaptor system coupled to the endoscope body and the endoscope neck slidably engaged with the adaptor rod via snaps in accordance with an embodiment.

FIG. 4 is an exemplary side view illustrating an endoscope adaptor system including a hinge between the rod and the coupling body to enable articulation of the rod in accordance with an embodiment.

FIG. 5 is an exemplary side view illustrating an endoscope adaptor system including attachment elements disposed along the rod for coupling auxiliary tools in accordance with an embodiment.

FIG. 6a is an exemplary perspective view illustrating an endoscope adaptor system including a hollow tubular rod with an entry opening and an exit port for receiving an endoscope neck in accordance with an embodiment.

FIG. 6b is an exemplary side view illustrating the endoscope adaptor system of FIG. 6a with a hollow tubular rod defining a channel for a flexible endoscope neck in accordance with an embodiment.

FIG. 7a is an exemplary first side view illustrating an endoscope system with an integral endoscope adaptor system having a hinged rod coupled to the endoscope body in accordance with an embodiment.

FIG. 7b is an exemplary second side view illustrating the endoscope system of FIG. 7a with the rod in a different articulated configuration in accordance with an embodiment.

FIG. 8 is an exemplary side view illustrating an endoscope system with an integral adaptor system, where the endoscope neck is coupled to the rod via snaps and the distal head is shown in a curled configuration in accordance with an embodiment.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION

Various embodiments shown and described herein include a universal endoscope adaptor system designed to be retrofitted to some or all leading-brand flexible endoscopic systems, or to other endoscopic systems, for use in otolaryngology and other surgical fields and for various other suitable uses. Flexible endoscopes can require both hands: one hand to hold the endoscope and operate a switch to change its direction, while the other hand stabilizes the scope and controls its forward and backward movement. Various embodiments discussed herein can address this limitation by offering a solution that allows the operator to control the flexible endoscope using just one hand, without the need to disassemble or reassemble the device.

An endoscope adaptor system in various embodiments can be configured to stabilize a flexible endoscope in the desired position, enabling the operator to simultaneously hold the scope, adjust the existing native directional switch of the endoscope, and control the forward-backward movement all with the same hand. This innovation, in various examples, frees the operator’s other hand to utilize additional surgical instruments, improving efficiency and precision during procedures and can reduce the need for an entirely different rigid endoscopy setup. The endoscope adaptor system in various embodiments transforms the flexible endoscope into a hybrid system that combines the stability of a rigid endoscope with the flexibility to alter the viewing angle and scope length on demand. Such a dual functionality in various examples not only enhances procedural versatility but also enables the visualization of anatomical regions that may be difficult or impossible to access using a rigid endoscope alone.

Unlike some systems where the handle merely holds the flexible scope in place without providing space for the same hand to operate it, various embodiments are configured to allow the operator to use their thumb to pivot the scope up and down while maintaining a secure grip. This integrated design in some examples can provide for smooth, continuous control of the endoscope’s movement without needing to switch hands or lose stability, enabling more precise navigation during procedures.

Various embodiments are configured to attach seamlessly to a grip of a flexible endoscope and/or the tip of the endoscope. In various examples, the system is configured to be easily removed or reattached during any stage of a procedure—before, during, or after—with no interruption to the procedure. The attachment in some embodiments can be adjusted with a posterior strap or clasp, to universally fit some or all endoscopes. Various embodiments require no additional power sources, electronic components, or modifications to the endoscope’s material properties, making various examples universally compatible across different endoscopic platforms and in different clinical environments or resource-poor areas.

Another embodiment may integrate the adaptor within the body of the endoscope itself. In such cases, the user could flip the adaptor outwards from the body to utilize it or choose to keep it hidden within the sheath of the body of the endoscope.

Some embodiments can have one or more of the following features:

Universal Compatibility: Unlike some systems that require specific endoscopic brands or models, various examples are configured to work with some or all leading flexible endoscope systems, making it versatile and cost-effective for healthcare institutions.

One-Handed Control: An endoscope adaptor system in various embodiments allows for quick conversion into a one-handed operation, dramatically reducing the operator’s physical burden while increasing precision and control during complex procedures. When a flexible scope is then needed to further investigate areas (e.g., go into the larynx, etc.) the endoscope adaptor system in some examples can simply be removed for easy conversion back into a flexible endoscope.

Quick Assembly/Disassembly: An endoscope adaptor system in various embodiments can be attached and/or removed without any specialized tools and/or without disrupting the sterility of the surgical field. In some embodiments, an endoscope adaptor system can be flipped outwards from the body on an as-needed basis.

Hybrid System: Various examples can provide the advantages of both rigid and flexible endoscopy by providing stability while retaining flexibility for enhanced navigation.

Cost effective: Rather than purchasing two different systems of scopes, monitors, light cords, etc. just the flexible endoscope with an endoscope adaptor system is now feasible in accordance with various embodiments. Furthermore, while some examples include a unique endoscope system that can toggle between flexible and rigid, an endoscope adaptor system of some examples can be universally applicable and alleviates the need to purchase a completely new endoscopy set up.

Various examples can include attachment points for auxiliary tools such as biopsy test tubes, suction catheters, swabs, balloon dilators, and the like. These integrated accessory points can streamline the procedure, allowing the operator to switch tools rapidly without having to interrupt the procedure to attach new instruments.

While some hybrid flexible-rigid designs require specific pre-existing equipment or systems to function, various embodiments can be partially or fully universal and can work independently or agnostically of any particular endoscopic setup. Various examples can be easily attached and removed without the need for modification to an existing flexible endoscope system. Once the endoscope adaptor system is removed, in various embodiments the endoscope can return to its original function without any adjustments, ensuring seamless transitions between traditional and hybrid modes of operation. This adaptability in some examples can allow the operator full control and flexibility during procedures, enhancing both usability and efficiency.

Various embodiments can comprise various suitable materials including metal, plastic, wood, or the like. Some examples can be made of materials that can be autoclaved and can be reusable. In some embodiments, the endoscope adaptor system is configured to not be reusable, but rather developed as a one-time use disposable device.

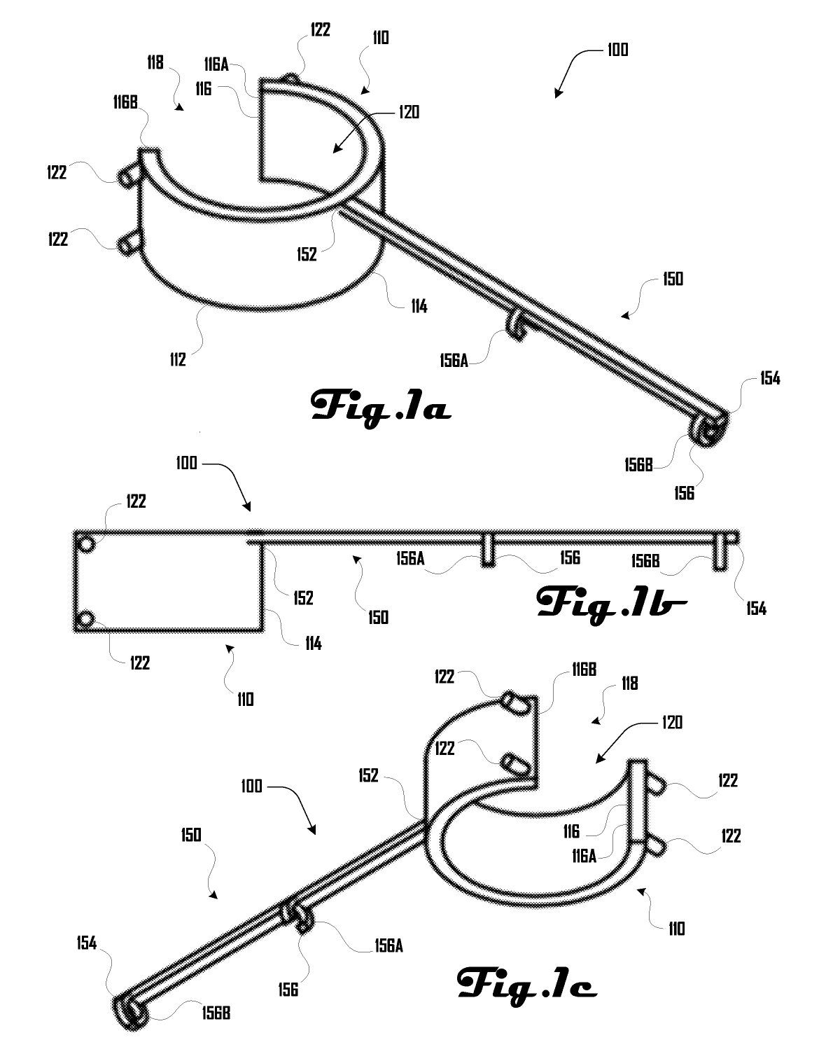

Turning to FIGS. 1a, 1b, and 1c, an example embodiment of an endoscope adaptor system 100 is illustrated, which can comprise, consist essentially of, or consist of a coupling portion 110 and a rod 150. Specifically, FIG. 1a illustrates a first perspective view of an endoscope adaptor system 100, FIG. 1c illustrates a second perspective view of the endoscope adaptor system 100, and FIG. 1b illustrates a side view of the endoscope adaptor system 100.

In various embodiments, the coupling portion 110 can comprise a coupling body 112 that has a front end 114 where the rod 150 is coupled to and extends from the front end 114 of the coupling body 112 as discussed herein. The coupling body 112 can extend to a pair of rear ends 116A, 116B that defines a gap 118, which defines an opening into a coupling cavity 120 defined by the coupling body 112.

In various embodiments, a coupling body 112 of a coupling portion 110 can comprise an adjustable, semicircular attachment point where the endoscope adaptor system 100 attaches securely to an existing flexible endoscope body, endoscope neck and/or endoscope tip. The coupling body 112 can have various suitable dimensions. For example, the coupling body 112 can have a thickness between a top and bottom face of 1.0 cm, 1.5 cm, 2.0 cm, 2.5 cm, 3.0 cm, 3.5 cm, 4.0 cm, 4.5 cm, 5.0 cm, 5.5 cm, 6.0 cm, or the like, or a range between such example values. In some embodiments, the coupling body can have a diameter of 3.0 cm, 3.5 cm, 4.0 cm, 4.5 cm, 5.0 cm, 5.5 cm, 6.0 cm, 6.5 cm, 7.0 cm, 7.5 cm, 8.0 cm, or the like, or a range between such example values. In various embodiments, the distance between the ends 116A, 116B that defines the gap 118 can be 1.0 cm, 1.25 cm, 1.5 cm, 1.75 cm, 2.0 cm, 2.25 cm, 2.5 cm, 2.75 cm, 3.0 cm, 3.25 cm, 3.5 cm, 3.75 cm, 4.0 cm, or the like, or a range between such example values. In some preferred embodiments, the width of the rod 150 can be between 0.25-1 cm and in one preferred embodiment 0.45 cm. In some preferred embodiments, the height of the rod 150 can be between 0.1-0.5 cm and in one preferred embodiment 0.2 cm. In some preferred embodiments, the length of the rod 150 can be between 2-30cm and in one preferred embodiment 9.5cm. In some preferred embodiments, the depth/diameter of the coupling body can be between 1-15 cm and in one preferred embodiment 4 cm. In some preferred embodiments, the distance of the gap 118 can be between 1-8cm and in one preferred embodiment 2cm. In some preferred embodiments, the thickness of the coupling body 112 can be between 0.5-10cm and in one preferred embodiment 2cm.

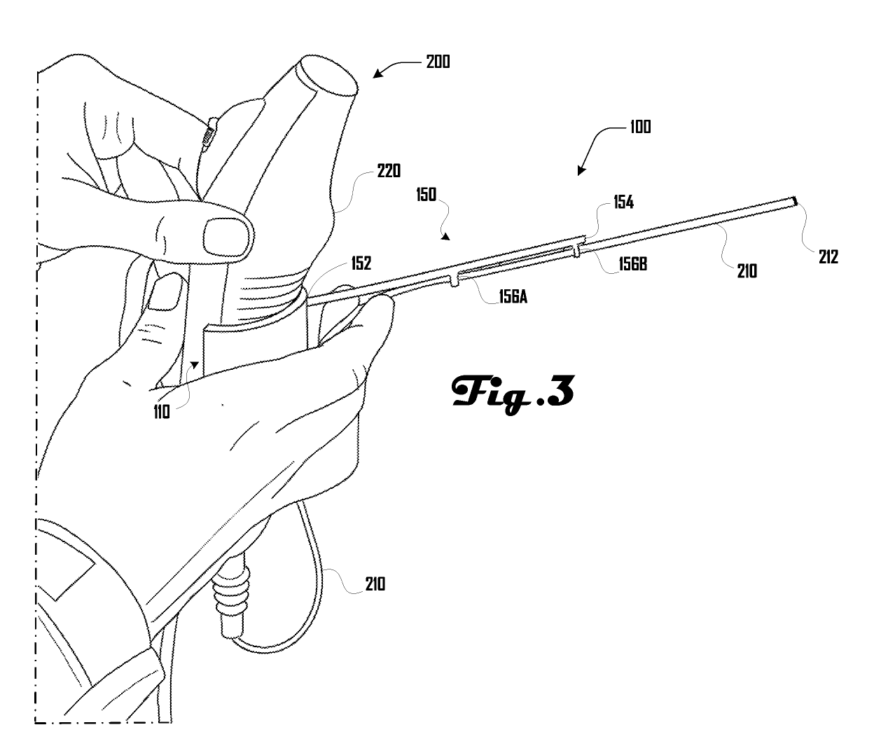

In various embodiments, the dimensions and shapes of the coupling body 112 can be configured to correspond to the size of the body of an endoscope, configured to be securely coupled with the body of an endoscope, configured based on the flexibility of the materials that define the coupling body or the like, as discussed in more detail herein. For example, FIG. 3 illustrates an example of a coupling body 112 of a coupling portion 110 of an endoscope adaptor system 100 coupled to the endoscope body 220 of an endoscope 200.

In some embodiments, an internal face of the coupling body 112 facing the coupling cavity 120 can be cushioned to assist with securely attaching to the endoscope body 220 of an endoscope 200 (see e.g., FIG. 3) and to prevent slippage or surface damage. For example, in some embodiments, a cushioning material can comprise a compliant or semi-compliant layer such as silicone rubber, thermoplastic elastomer (TPE), polyurethane foam, silicone gel, natural or synthetic rubber, or a polymeric elastomer laminate. In some embodiments, the cushioning material has a durometer in a range configured to provide sufficient radial compressive force and frictional resistance to movement when the coupling body 112 is installed, such as between about 20A and 80A on the Shore scale. In further embodiments, the cushioned layer may be textured, ribbed, dimpled, or coated with a high-friction or tackified surface treatment to enhance grip against the outer sheath of the endoscope.

In certain embodiments, the cushioning material may be adhesively bonded, mechanically retained, or co-molded within the coupling cavity 120. In other embodiments, the internal face may include a replaceable or disposable insert, liner, or sleeve to maintain sterility, accommodate endoscopes of different diameters, or provide variable frictional performance. The cushioned interface can also serve to dampen vibration and reduce localized stress while allowing the physician to selectively adjust the rigidity of the composite endoscope-coupling assembly by positioning or removing the coupling body 112 along the endoscope shaft. In this manner, a single flexible endoscope may be selectively converted between flexible and rigid states or may be configured to provide graduated stiffness along its length, thereby improving clinical access, stability, and precision in a range of diagnostic and therapeutic procedures.

In various embodiments, the dimensions and shapes of the coupling body 112 can be configured to correspond to the size and external geometry of the endoscope body 220 of an endoscope 200 (see e.g., FIG. 3). For example, the coupling body 112 may define a longitudinal channel, lumen, or coupling cavity 120 configured to receive a portion of a flexible endoscope shaft. The coupling body 112 can be configured to interface with commercially available flexible endoscopes of various diameters, lengths, and construction materials. In some embodiments, the coupling body 112 is formed from a relatively rigid polymer, metal, composite, or multi-material assembly that, when operably engaged with the endoscope, constrains bending of the portion of the endoscope inserted into the coupling cavity 120, thereby selectively converting that endoscope portion from a flexible configuration to a substantially rigid configuration while maintaining the functionality of the distal optics and instrumentation of the endoscope.

The coupling body 112 may be dimensioned and shaped based on the flexibility, modulus, and deformation characteristics of the materials used to define the coupling body 112, such that the coupling body 112 can securely receive and retain the endoscope without damaging or permanently deforming the endoscope shaft. In particular, the coupling cavity 120 may be sized to achieve an interference fit with the endoscope shaft, a clamping interface, a snap-fit or bayonet-fit interface, or other mechanical securing configuration. The coupling body 112 may further include one or more slits, slots, or cut-outs, allowing the coupling body 112 to elastically expand or contract to accommodate endoscopes of slightly different diameters while maintaining a secure engagement.

In some example embodiments discussed herein, the coupling body 112 may define a generally C-shaped configuration when viewed in cross-section, facilitating lateral installation onto a flexible endoscope shaft and allowing the coupling body 112 to resiliently expand or flex to accommodate the endoscope diameter. However, this C-shaped configuration represents only one exemplary embodiment and is not intended to limit the scope of the present disclosure.

For example, in some embodiments, the coupling body 112 may be generally U-shaped, J-shaped, horseshoe-shaped, or arcuate with asymmetrical arms such that one rear end 116A extends further than the other rear end 116B. These configurations may facilitate one-handed installation, orientation-specific mounting, or controlled retention forces along particular contact regions of the endoscope shaft. In other embodiments, the coupling body 112 may be O-shaped or fully annular, defining a closed loop or tubular channel into which a distal section of the endoscope can be inserted axially. A split-ring configuration, hinged ring configuration, quick-clamp ring, or latchable annular body may also be used in some examples to combine the advantages of circumferential support with ease of placement and removal.

In some embodiments, the coupling body 112 can define polygonal, elliptical, oval, or semi-elliptical internal and/or external profiles, such as D-shaped, trapezoidal, hexagonal, or faceted geometries. These variations may be selected to interface with specific endoscope designs, to improve torsional stability, to accommodate integrated cables or lumens on one side of the endoscope shaft, or to enhance ergonomic manipulation by the operator. In some embodiments, the coupling cavity 120 cross-section may be non-uniform or tapered, providing graded stiffness characteristics along the length of the coupling body 112 or allowing selective axial positioning to tune rigidity of the endoscope.

Some embodiments may include modular or segmented coupling bodies 112 comprising multiple interlocking components, such as complementary C-shaped halves, dovetail-engaged arcuate pieces, or rail-mounted rigidizing elements that are attachable along selected portions of the endoscope shaft. In some such configurations, the gap 118 may be adjustable in width, angular orientation, or closure force, for example via threaded fasteners, ratcheting bands, sliding collars, biased spring arms, shape-memory elements, or cam-locking mechanisms. Such modular designs may enable a clinician to progressively build a composite semi-rigid or fully rigid support structure around a flexible endoscope at varying lengths and positions, depending on procedural demands.

In some embodiments, the coupling body 112 may incorporate one or more integrated flexure zones, living hinges, or compliant hinge segments that allow the coupling body 112 to momentarily open, expand, or pivot to receive the endoscope shaft and subsequently return toward a closed or semi-closed configuration to secure the shaft. Some such configurations may be advantageous for rapid deployment, secure repositioning along the endoscope during use, and accommodating varying shaft diameters while maintaining axial and torsional rigidity during operation.

Returning to the examples of FIGS. 1a, 1b and 1c, four posts 122 line the exterior face of the rear ends 116A, 116B of the coupling body 112 (e.g., two on each end 116) that can serve as attachment points for one or more flexible straps, bands, or the like, that can be tethered across the gap 118 and around an endoscope to provide additional coupling support if needed or desired. Although flexible straps or bands are shown and discussed as one exemplary mechanism for stabilizing the coupling body 112 relative to an endoscope, various other support structures, tension-generating members, and retention elements may be incorporated without departing from the scope or spirit of the present disclosure.

For example, in some embodiments, the coupling body 112 may include one or more integrated tension bands, elastic rings, or circumferential collars configured to span across the gap 118 and exert inward radial pressure around a received endoscope shaft. The tensioning members may include elastic silicone bands, thermoplastic elastomer (TPE) rings, braided polymer cinch straps, adjustable hook-and-loop retention straps, or pre-curved spring-steel clips configured to snap into engagement with the posts 122 or complementary engagement features on the coupling body 112. In certain embodiments, the tensioning mechanism may include a ratcheting strap system, buckle mechanism, cam-lever clamp, or miniature cable-tie device configured to draw the rear ends 116A, 116B toward one another to ensure a frictional fit and secure retention of the endoscope.

In further embodiments, the coupling body 112 can incorporate a semi-rigid over-center clamp, hinged closure arm, or locking ring configured to pivot or slide across the gap 118. Such mechanisms may be rotationally or linearly actuated to fully or partially close the gap 118, thereby enabling the user to selectively increase the rigidity and holding force. In yet other embodiments, a flexible compression sleeve, shrink-fit collar, or reversible elastic sheath may be positioned over the coupling body 112 after the endoscope is inserted into the coupling cavity 120, thereby providing 360-degree circumferential support and stabilizing the interface.

In some embodiments, magnetic, adhesive, or suction-assisted retention structures may also be used to supplement or replace mechanical fasteners. For example, the rear ends 116A, 116B in some examples can include embedded magnets configured to attract one another or interface with a ferromagnetic retaining strap. In some examples, low-tack biocompatible adhesive pads, micro-suction polymer films, or vacuum-actuated flexible bladders may be positioned on or adjacent the posts 122 to improve stability of the endoscope adaptor system 100. In some embodiments, the coupling body 112 can further include a modular auxiliary clamp or sliding collar positioned along the rod 150 and configured to cinch the coupling body 112 against the outer surface of the endoscope for enhanced retention.

These alternative structures and mechanisms may be used independently or in combination to tailor the coupling force, rotational stability, axial retention, or rigidity characteristics of the coupling body 112 during endoscopic procedures. Additionally, such supplemental support mechanisms may be disposable, sterilizable, integrated, removable, interchangeable, or user-selectable depending on procedural requirements, endoscope size, operator preference, or the like.

Returning to the examples of FIGS. 1a, 1b, and 1c, the rod 150 is coupled to and extends from the front end 114 of the coupling body 112 (e.g., from a rod base 152 to a rod end 154). In various embodiments, the rod 150 can extend from the front end 114 parallel to a main axis of the coupling body 112 and/or parallel to one or both of top and bottom faces of the coupling body 112. In certain embodiments, the rod 150 can have a linear rectangular shape; however, this configuration is merely exemplary and should not be construed as limiting, as the rod 150 can assume any suitable cross-sectional profile or geometric configuration consistent with the intended structural and functional objectives described herein (e.g., cylindrical).

In some embodiments, the rod 150 may have a non-circular cross section such as oval, elliptical, rectangular, square, polygonal, or D-shaped, which can provide increased torsional stiffness, anti-rotation features, or improved grip for manual manipulation. In other embodiments, the rod 150 may define a flattened, ribbon-like, or blade-shaped profile to minimize overall thickness and weight while maintaining rigidity in one or more desired directions. Such profiles can be particularly advantageous when the coupling portion 110 is intended to lie close to a patient surface or within a constrained surgical workspace.

In further embodiments, the rod 150 may have a tubular or hollow configuration, reducing weight while enabling the passage of control cables, electrical leads, or fluid conduits therethrough. The inner lumen of such a tubular rod may also house a fiber-optic line, electrical conductor, sensor wire, or other functional component. In other embodiments, the rod 150 may include an I-beam, C-channel, or truss-like structure to optimize strength-to-weight ratio and resist bending or torsional deformation.

In still further embodiments, the rod 150 may include one or more surface features, such as longitudinal ribs, flutes, grooves, splines, or knurled textures, configured to interface with mating clamps, linear guides, or sliding collars that provide adjustable positioning. In some variations, the rod 150 may include a telescoping segment, flexible joint, or hinge section that allows the operator to selectively vary its length or orientation while maintaining the ability to transmit mechanical loads between the coupling body 112 and other support structures.

Collectively, these alternative rod shapes and cross-sectional geometries in some embodiments can enable tailoring of stiffness, weight, manufacturability, and functional integration (e.g., allowing the coupling portion 110 to be optimized for different endoscope types, procedural environments, and operator preferences) while remaining within the scope and spirit of the present disclosure.

In various embodiments, the rod 150 can have various suitable lengths (e.g., from the rod base 152 to the rod end 154) including 8.0 cm, 9.0 cm, 10.0 cm, 11.0 cm, 12.0 cm, 13.0 cm, 14.0 cm, 15.0 cm, 16.0 cm, 17.0 cm, 18.0 cm, 19.0 cm, 20.0 cm, 21.0 cm, 22.0 cm, 23.0 cm, 24.0 cm, 25.0 cm, 26.0 cm, 27.0 cm, 28.0 cm, 29.0 cm, 30.0 cm, 35.0 cm, 40.0 cm, 50.0 cm or the like, or a range between such example values. In various embodiments, the rod 150 can have various suitable diameters or maximum widths, such as 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, or the like, or a range between such example values.

In various embodiments, the rod 150 can extend from the front end 114 parallel to a main axis of the coupling body 112 and/or parallel to one or both of top and bottom faces of the coupling body 112, which can be defined as an angle of 0º. However, in some embodiments the rod 150 can extend at any suitable angle such as -35º, -30º, -25º, -20º, -15º, -10º, -5º, 0º, 5º, 10º, 15º, 20º, 25º, 30º, 35º, or the like, or a range between such example values.

In various embodiments, the rod 150 can have a plurality of snaps 156 along the length of the rod 150. For example, as shown in the examples of FIGS. 1a, 1b, and 1c, the rod 150 can have a first snap 156A disposed between the rod base 152 and rod end 154 (e.g., half way between) and a second snap 156 disposed at or proximate to the rod end 154. In various embodiments, the snaps 156 can extend from the rod 150 the same direction. Also, while various examples discussed herein can include two snaps 156, it should be clear that further embodiments can include a single snap 156 or any suitable plurality of snaps such as 1, 2, 3, 4, 5, 6, 7, 8, 9, 10, 12, 15, 20, 25, or the like or a range between such example values.

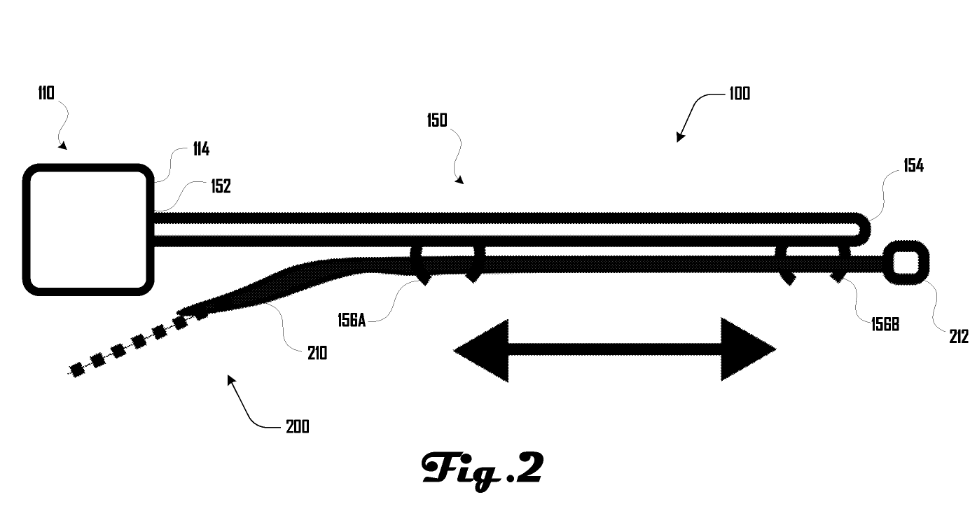

In various embodiments, one or more snaps 156 can be configured to hold a flexible neck of an endoscope in place and can give structure and rigidity to the flexible neck of the endoscope, but can be configured to provide enough space such that the user can move the scope forwards and backwards using the same hand that is holding the endoscope body of an endoscope. For example, FIG. 2 illustrates a side view of an example embodiment of an endoscope adaptor system 100 of one embodiment where an endoscope 200 is coupled with the endoscope adaptor system 100 including a neck 210 of the endoscope 200 extending through a first and second snap 156A, 156B such that the neck 210 of the endoscope 200 is slidably held within the snaps 156 and with a head 212 of the endoscope 200 extending through the second snap 156B at the rod end 154 and with the head 212 of the endoscope 200 extending past the rod end 154. In various embodiments, the coupling of the snaps 156 and the neck 210 of the endoscope 200 can allow for bi-directional movement of the neck 210 of the endoscope 200 along the length of the rod 150 as discussed herein.

In various embodiments, the coupling body 112 of the endoscope adaptor system 100 can be coupled with a body 220 of the endoscope 200 as discussed herein (see e.g., FIG. 3) such that the snaps 156 point downward and such that the neck 210 of the endoscope 200 is disposed below the rod 150, which can be desirable in some examples by allowing the neck 210 of the endoscope to hang from the snaps 156 below the rod 150. However, in some embodiments, the neck 210 of the endoscope 200 can be configured to be disposed above or to the side of the rod 150.

The length of the rod 150 in various embodiments can be variable and can be extended partially or completely over the length of the rod 150 to allow flexibility in how much of the endoscope neck 210 is inserted (e.g., into a sinonasal cavity). The stabilizing rod can be columnar in shape or rectangular with beveled edges in some examples. The stabilizing rod can be made of a fully rigid material or with varying flexibility and memory to allow for angulation of the endoscope. The snaps 156 in various embodiments are integrally or movably attached to the rod 150 and can be either conjoined at the inferior aspect or apart at various angles. The snaps 156 may be of various thicknesses and number of snaps 156 can be selected in some examples depending on the length of the rod 150.

While some embodiments employ generally C-shaped snaps 156, various suitable alternative coupling configurations may be used to support or guide the flexible neck 210 of the endoscope 200 relative to the rod 150. For example, the coupling structures 156 may comprise a closed circle where the head 212 and neck 210 of the endoscope 200 are threaded through, U-shaped clips, semi-circular clamps, hinged rings, or arcuate collars configured to partially or fully surround the neck 210. In some embodiments, the coupling structures 156 may be D-shaped, elliptical, rectangular, polygonal, or slotted to accommodate endoscope necks 210 of various diameters and geometries. Other examples may include resilient loop members, elastomeric bands, or spring-biased jaws configured to grip or cradle the neck 210 while allowing axial sliding movement along the rod 150. In further embodiments, the snaps 156 may include hook-and-loop or detent-type mechanisms, sliding sleeves, magnetic clips, or compliant saddles formed from polymeric or silicone materials to reduce friction and prevent abrasion of the endoscope neck 210.

In some embodiments, the coupling structures 156 can be oriented such that the neck 210 of the endoscope 200 hangs beneath the rod 150, as shown in FIG. 2, which can provide ergonomic and gravitational stabilization. However, other configurations of further embodiments can include snaps 156 that are arranged to support the endoscope neck 210 above or laterally beside the rod 150. For example, the snaps 156 may be rotatable or reversible in some examples, allowing the user to reposition the endoscope orientation for specific clinical approaches or to accommodate different working angles. The coupling structures 156 may also include offset brackets or adjustable mounts enabling the neck 210 to be held at a spaced distance from the rod 150, providing clearance for integrated illumination cables, suction conduits, or other accessories.

The coupling structures 156 may be integrally formed with the rod 150 (e.g., via molding or additive manufacturing) or removably attached thereto by fasteners, dovetail slides, rail systems, or snap-fit connections. In certain embodiments, the coupling structures 156 can include hinge joints, flexible arms, or spring-steel clips enabling the coupling element to open and close about the neck 210 for quick installation and removal. In some examples, a continuous or segmented guide channel may be provided along the rod 150, within which the neck 210 is received and restrained by an overlying flexible strap, elastic sheath, or sliding retainer bar.

The snaps 156 may further incorporate friction-modifying features, such as low-friction polymer liners, PTFE or silicone coatings, or embedded rolling elements to facilitate smooth bi-directional movement of the endoscope neck 210 relative to the rod 150. In still further embodiments, the snaps 156 may include locking or detent mechanisms configured to temporarily secure the endoscope neck 210 in a desired position along the rod 150 during use and release it upon manual actuation. Collectively, these alternative configurations allow the endoscope adaptor system 100 of various embodiments to accommodate a wide range of sizes of endoscope necks 210, stiffness levels, and procedural orientations while maintaining stable yet adjustable coupling between the rod 150 and the endoscope neck 210.

FIG. 3 illustrates an example of a user holding the endoscope body 220 of an endoscope 200 where an endoscope adaptor system 100 is coupled to the endoscope body 220 of an endoscope 200. Additionally, the neck 210 of the endoscope 200 is shown slidably coupled to the rod 150 of the endoscope adaptor system 100 via first and second snaps 156A, 156B disposed along the length of the rod 150.

In various embodiments, the endoscope adaptor system 100 is configured to allow the user to move the neck 210 of the endoscope 200 along the length of the rod 150, and to extend the head 212 of the neck 210 a desired length past the rod end 154, with fingers of the same hand holding the endoscope body 220 of the endoscope 200. For example, an endoscope adaptor system 100 in various embodiments can be configured to stabilize a flexible endoscope neck 210 of an endoscope in a desired position, enabling the operator to simultaneously hold the body 220 of the endoscope 200, adjust the existing native directional switch of the endoscope, and control the forward-backward movement of the neck 210 all with the same hand. Accordingly, the endoscope adaptor system 100 in various examples, frees the operator’s other hand to utilize additional surgical instruments or perform additional tasks, which can improve efficiency and precision during procedures and can reduce the need for an entirely different rigid endoscopy set up. The endoscope adaptor system 100 in various embodiments transforms the flexible endoscope neck 210 into a hybrid system that combines the stability of a rigid endoscope with the flexibility to alter the viewing angle and scope length on demand. Such a dual functionality in various examples not only enhances procedural versatility but also enables the visualization of anatomical regions that may be difficult or impossible to access using a rigid endoscope alone.

In various embodiments, the head 212 of the neck 210 can be configured to extend various suitable lengths past the rod end 154 of the rod 150, including in some examples at least including 1.0 cm, 2.0 cm, 3.0 cm, 4.0 cm, 5.0 cm, 6.0 cm, 7.0 cm, 8.0 cm, 9.0 cm, 10.0 cm, 11.0 cm, 12.0 cm, 13.0 cm, 14.0 cm, 15.0 cm, 16.0 cm, 17.0 cm, 18.0 cm, 19.0 cm, 20.0 cm, 21.0 cm, 22.0 cm, 23.0 cm, 24.0 cm, 25.0 cm, 26.0 cm, 27.0 cm, 28.0 cm, 29.0 cm, 30.0 cm, or the like, or a range between such example values.

In various embodiments, a method of using an endoscope can include coupling an endoscope adaptor system 100 to the endoscope body 220 of an endoscope 200 by securing a coupling body 110 of the endoscope adaptor system 100 to the endoscope body 220 of the endoscope 200. In some examples, the coupling body 110 can be further secured to the endoscope body 220 in various suitable ways such as coupling flexible straps, bands, or the like, to one or more posts 122 of the coupling body 110, which can be tethered across a gap 118 of the coupling body 110 and around the endoscope body 220 to provide additional coupling support to the coupling body 110 on the endoscope body 220.

Coupling the coupling body 110 to the endoscope body 220 can cause a rod 150 of the endoscope adaptor system 100 to extend from the endoscope body 220 and the endoscope neck 210 can be slidably coupled to the rod 150. For example, in some embodiments, the coupling body 110 can be attached to the endoscope body 220 with a plurality of C-shaped or O-shaped snaps 156 extending downward from the rod 150 and the endoscope neck 210 can be coupled to the rod 150 by inserting the neck 210 into or through the snaps 156 with a head 212 of the neck 210 extending past a distal rod end 154 of the rod 150.

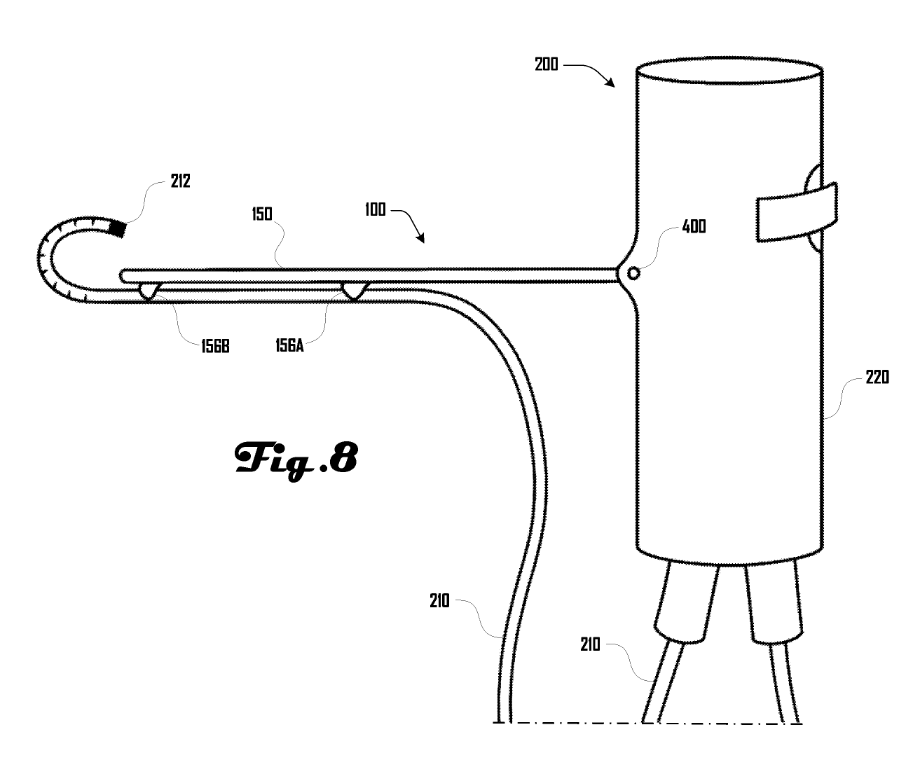

The user can then grasp the endoscope body 220 of the endoscope 200 with one hand and can manipulate the neck 210 of the endoscope 200 to move the neck 210 along the length of the rod 150, and to extend and/or retract the head 212 of the neck 210 a desired length past the rod end 154, with fingers of the same hand holding the endoscope body 220 of the endoscope 200. Fingers of the same hand can actuate a button or trigger of the endoscope 200 to change the direction of the head 212 of the endoscope 200 (see e.g., FIG. 8). In various examples, this can allow the user to simultaneously hold the endoscope 200, adjust the existing native directional switch of the endoscope 200 to change the configuration of the head 212 and distal portion of the neck 210, and to control the forward-backward movement of the neck 210 all with the same hand.

While some embodiments of an endoscope adaptor system 100 provide for only manual actuation and lack components such as a power source, motor, or the like, some embodiments can allow for motorized or electromechanical actuation of the endoscope neck 210 to cause extension and/or retraction of the head 212 of the neck 210 a desired length past the rod end 154.

For example, in some embodiments, the rod 150 or coupling body 110 can house one or more linear actuators, servo motors, or stepper motors coupled to a carriage or clamp that engages the neck 210. The motorized actuator can advance or retract the carriage along the longitudinal axis of the rod 150, thereby driving controlled forward or backward motion of the neck 210 and selectively positioning the head 212 of the endoscope 200 relative to the distal rod end 154. In certain embodiments, the actuator may include a linear screw drive, rack-and-pinion system, lead-screw assembly, or toothed-belt and pulley arrangement, each configured to translate rotary motion of the motor into linear displacement of the neck 210.

In some embodiments, one or more motors may be integrated into the rod 150 itself or disposed within a housing coupled to the coupling body 110. Electrical power for such actuators can be provided via an onboard rechargeable battery, an external power connector, or inductive coupling from a wireless charging base. The motor control signals may be delivered through wired connections extending along the rod 150 or transmitted wirelessly using Bluetooth, Wi-Fi, or near-field communication (NFC) protocols. A control interface—such as a thumb-operated toggle, rocker switch, trigger, or capacitive touchpad—may be positioned on or near the endoscope body 220 or coupling body 110, allowing the operator to control extension or retraction with the same hand that holds the endoscope.

In further embodiments, a microcontroller or embedded processor may coordinate the operation of the motorized mechanism based on positional feedback from one or more sensors. For example, the rod 150 may incorporate linear encoders, optical position sensors, Hall-effect sensors, or strain-gauge feedback elements that detect the real-time position or tension of the neck 210. The feedback can be used to modulate actuator speed, limit displacement, or enable pre-programmed extension depths for repeatable procedures. In some embodiments, the system may include haptic or visual feedback (e.g., vibration or indicator LEDs) to alert the user when the neck 210 has reached a preset travel limit or when an obstruction is detected.

In still other embodiments, the motorized actuation can be pneumatically or hydraulically driven rather than electromechanical. For instance, a pneumatic cylinder or hydraulic piston housed within the rod 150 can advance or retract the neck 210 based on user-controlled pressure changes. The control valve or pressure regulator may be integrated into the endoscope handle or operated remotely via a foot pedal or control console, providing sterile, hands-free control of endoscope extension.

In some embodiments, the motorized movement of the neck 210 may be coordinated with automated articulation of the head 212 or distal portion of the endoscope 200. For example, synchronized control software may enable compound movements in which forward extension of the neck 210 is accompanied by a specific deflection angle or rotation of the endoscope tip to maintain a constant field of view. Such systems may also include programmable presets, allowing automatic retraction to a safe position at the conclusion of a procedure, or slow-speed “follow” modes in which the endoscope advances or retracts proportionally to the user’s hand motion detected by inertial or proximity sensors.

Various embodiments can be implemented using modular motor units attachable to an otherwise manual adaptor system 100, or as fully integrated motorized adaptor assemblies. Materials for the drive components may include stainless steel or titanium for structural elements, reinforced polymers such as PEEK or polycarbonate for housing components, and medical-grade silicone or PTFE for friction-reducing sleeves contacting the endoscope neck 210. Various configurations can enable precise, repeatable, and ergonomic control of endoscope motion while maintaining compatibility with existing flexible endoscope systems and procedural workflows.

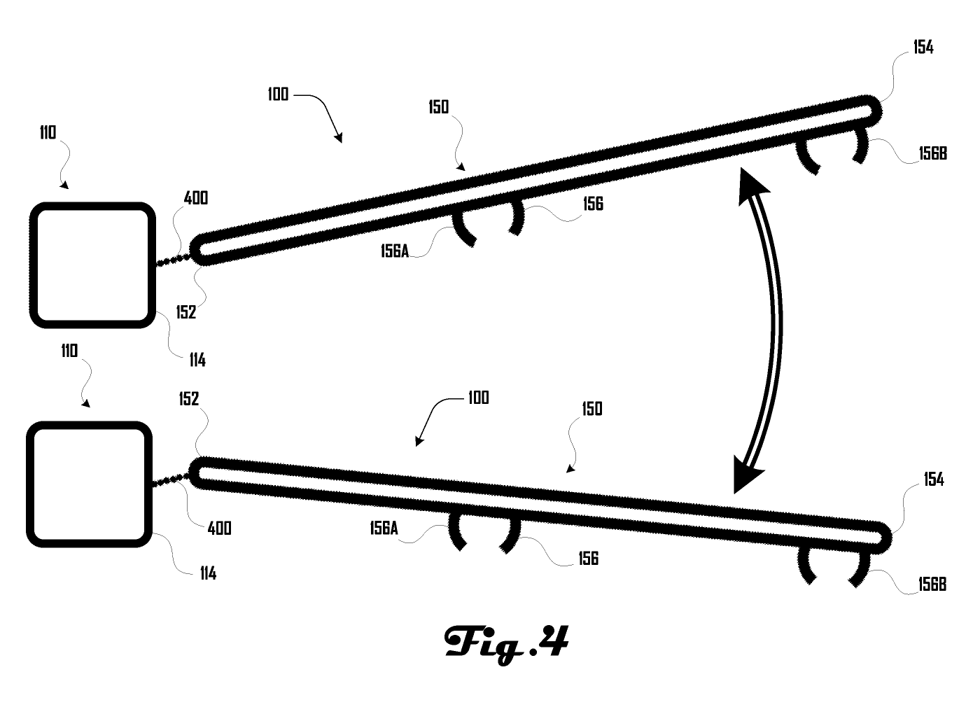

In some embodiments, the rod 150 can be rotatably or movably coupled to the coupling body 110. For example, FIG. 4 illustrates a side view of an example embodiment of an endoscope adaptor system 100 that comprises a hinge 400 disposed at the rod base 152 and at the front end 114 of the coupling body 112. In various embodiments, the hinge 400 can be configured to move up and down, laterally, or the like.

In some embodiments, the hinge 400 may include a simple mechanical pivot or pin joint formed by a pivot pin extending through aligned apertures in the coupling body 112 and the rod base 152. Such a configuration may allow the rod 150 to swing upward and downward relative to the coupling body 110 while maintaining axial alignment. In other embodiments, the hinge 400 may include a ball-and-socket joint, spherical bearing, or universal joint assembly configured to permit multi-axis articulation of the rod 150 relative to the coupling body 112. Some such embodiments can enable smooth, continuous adjustment in both vertical and lateral directions, allowing the operator to modify the field of motion of the coupled endoscope neck 210 without removing or repositioning the endoscope adaptor system 100.

In further embodiments, the hinge 400 may comprise a flexible or semi-rigid coupling section formed from an elastomeric material, a coiled spring segment, a living hinge, or a shape-memory polymer hinge. Such embodiments can provide controlled flexibility and positional memory, allowing the rod 150 to bend or deflect under user-applied force and return to a neutral orientation when released. In still other embodiments, the hinge 400 may include an articulating linkage or knuckle joint incorporating one or more interlocking plates, sliding rails, or telescoping arms to achieve compound angular or translational motion.

The hinge 400 in some embodiments can include locking or tensioning mechanisms configured to selectively fix or resist motion once a desired position of the rod 150 is achieved. For example, the hinge 400 may incorporate a friction washer, ratchet-lock assembly, detent clutch, threaded clamp, or cam-lock lever enabling the user to secure the rod 150 at a particular inclination or lateral offset. In certain embodiments, the hinge 400 may include a manually adjustable tightening screw, set screw, or spring-loaded ball detent to maintain stable angular positioning under operational loads.

In additional embodiments, the hinge 400 may be configured for lateral displacement of the rod 150 relative to the coupling body 110, such as through a sliding dovetail, linear bearing, or offset pivot assembly. Such motion can enable the user to reposition the endoscope neck 210 laterally in some examples for optimal access or visibility while maintaining a stable coupling to the endoscope body. Multi-axis hinge assemblies combining rotational, pivotal, and translational components are also present in some embodiments, allowing for both angular and positional adjustments in three dimensions.

Various hinge 400 embodiments can allow for controlled and repeatable articulation of the rod 150 relative to the coupling body 110, thereby increasing the ergonomic flexibility and functional adaptability of the endoscope adaptor system 100. The hinge 400 can be fabricated from biocompatible metals (e.g., stainless steel, titanium), rigid or semi-rigid polymers (e.g., acetal, PEEK, ABS), or composite materials depending on the desired mechanical performance, sterilization requirements, and manufacturing considerations.

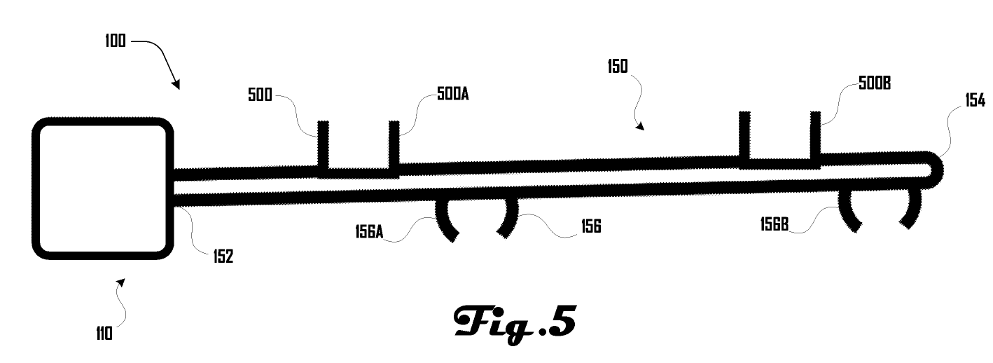

In various embodiments, an endoscope adaptor system 100 can include attachment elements for attaching various elements to the coupling body 110, rod 150, or the like. For example, FIG. 5 illustrates a side view of an embodiment of an endoscope adaptor system 100 that includes first and second attachment elements 500A, 500B disposed along the length of the rod 150 between the rod base 152 and the rod end 154. These attachments can be in various suitable angles respective to snaps 156 for holding a flexible endoscope neck 210. For example, as shown in FIG. 5, the attachment elements 500 can be disposed on an opposing side of the rod 150 from which the snaps 156 extend. For example, attachment elements 500 in some embodiments can extend superior or bilaterally perpendicular to the snaps 156 inferiorly. There can be any suitable number of attachment elements 500 (e.g., 1, 2, 3, 4, 5, 6, 7, 8, 9, 10, 12, 15, 20, and the like or a range between such example values). Attachment elements 500 can include various suitable elements, including in some embodiments, elements discussed herein related to various embodiments of snaps 156.

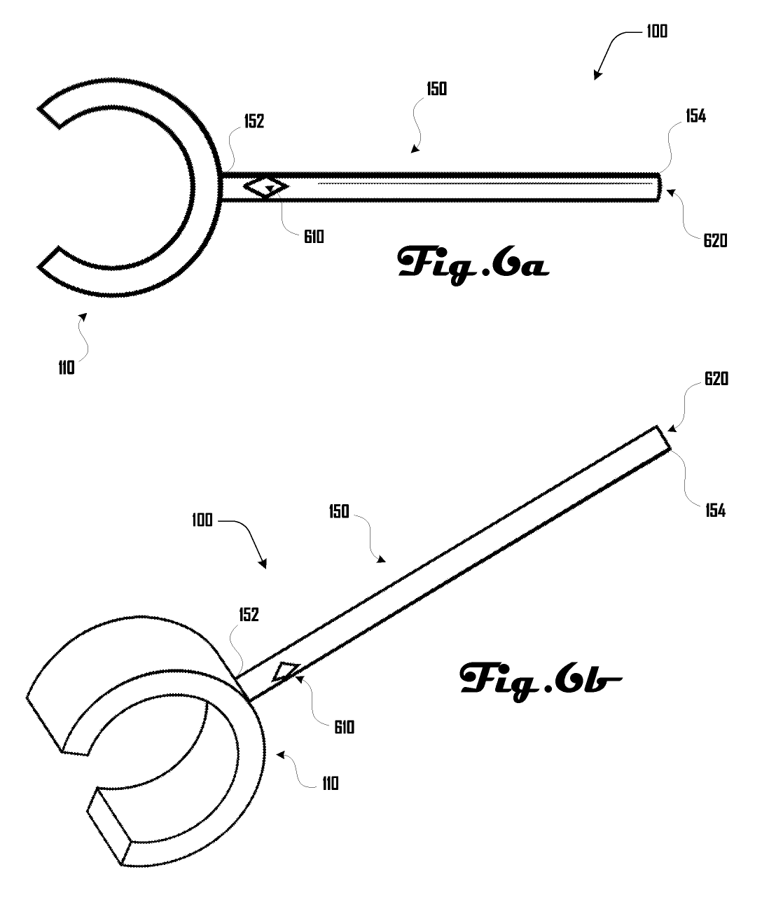

In various examples, the rod 150 of an endoscope adaptor system 100 can comprise a columnar hollow tube having a slightly larger diameter than the size of an endoscope neck 210. For example, FIGS. 6a and 6b illustrate an example embodiment of an endoscope adaptor system 100 that includes a rod 150 extending from a coupling body 110, where the rod 150 is defined at least in part by a hollow tube and comprises an entry opening 610 and an exit port 620 at the rod end 154.

For example, in various embodiments the head 212 of an endoscope neck 210 can be inserted into the entry opening 610 of the rod 150 and the endoscope neck 210 can be pushed through the hollow rod 150 until the head 212 of an endoscope neck 210 exits the exit port 620 at the rod end 154 of the rod 150. In various embodiments, pushing and pulling the endoscope neck 210 can cause the head 212 and distal end of the endoscope neck 210 to extend and retract from the exit port 620 to change the length of the endoscope neck 210 extending from the rod end 154 of the rod 150. The cavity of the rod 150 can have various suitable diameters such as a diameter in some examples of 3 mm or 8 mm, and in some examples 2 mm, 3 mm, 4 mm, 5 mm, 6 mm, 7 mm, 8 mm, 9 mm, 10 mm, 12 mm, 15 mm, 20 mm, or the like, or a range between such example values.

In some embodiments, a columnar hollow tube can be attached to a coupling body 110 via a hinge 400 (see e.g., FIG. 4) such as via a material with memory rigidity, or a hole and key model with the holes of various angles on the anterior aspect of the rod 150. In some embodiments, different rods 150 having different sized columnar hollow tubes can be configured to be modularly attached to a coupling body 110 so that different sized endoscope necks 210 can be used, so that different lengths of rods 150 can be used, and the like. Additionally, various different types of non-hollow rods 150 can be configured to be modularly coupled with coupling body 110.

In various embodiments a posterior inferior portion of the columnar tube of the rod 150 can define an entry opening 610 having an ellipses shape allowing passage of the endoscope from the inferior aspect into the columnar tube and finally out the exit port 620 at the rod end 154 of the columnar hollow tube of the rod 150. The entry opening 610 can be any suitable shaped hole and can be placed at any suitable location the length of the rod 150 (e.g., along the inferior aspect of the columnar hollow tube). This in various examples can allow safe passage of some or the entire columnar hollow tube into the body of a human or animal patient (e.g., the nasal cavity). For usage in some surgical specialties such as urology, the tube can also come in various suitable lengths as discussed herein. Additionally, in some embodiments, the entry opening 610 and/or exit port 620 can be configured to have a varying size so as to accommodate different sizes of endoscope necks 210.

In various embodiments, the system and/or components thereof are configured for an endoscope adaptor system 100 that largely remains outside of the body of a patient; however, some embodiments can be configured to be partially or fully inserted into the body of the patient.

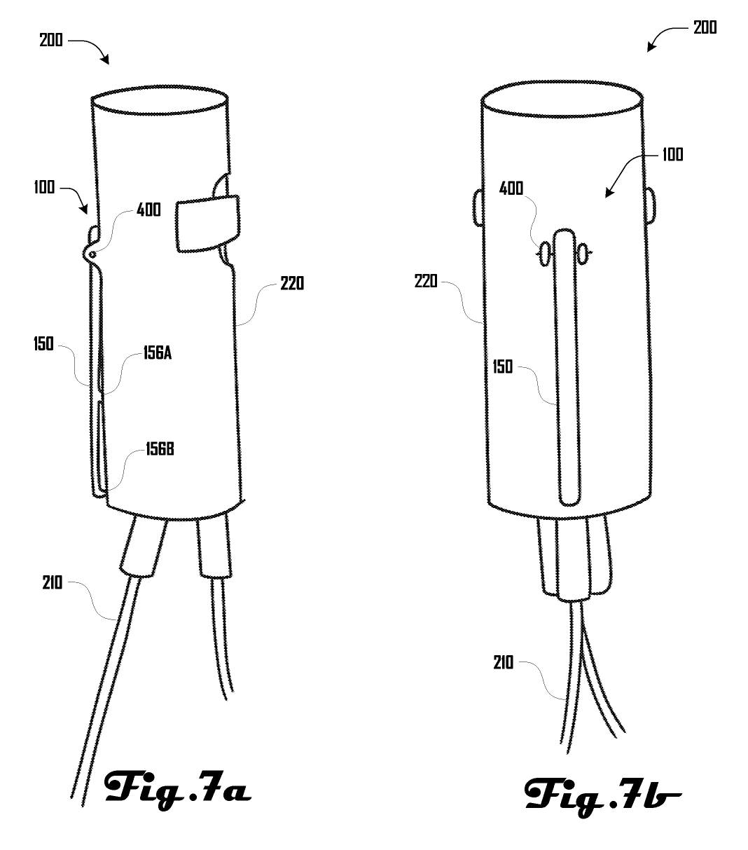

While various embodiments of an endoscope adaptor system 100 can be an after-market device configured to couple with various existing endoscope systems 200, some embodiments can include an endoscope adaptor system 100 that is integral to or a part of a commercial endoscope system 200 in contrast to an after-market device. For example, FIGS. 7a, 7b, and 8 illustrate example embodiments of an endoscope system 200 that comprises an integral endoscope adaptor system 100.

FIG. 7a illustrates a first side view of one embodiment that includes a rod 150 coupled to the endoscope body 220 of the endoscope 200 via a hinge 400, and FIG. 7b illustrates a second side view of the embodiment of FIG. 7a. FIG. 8 illustrates a side view of an embodiment that includes a rod 150 coupled to the endoscope body 220 of the endoscope 200 via a hinge 400, with the rod 150 shown in an extended configuration and with a neck 210 of the endoscope coupled to first and second snaps 156 disposed along the rod 150. The distal end of the endoscope neck 210 including the head 212 is shown actuated in a curled configuration.

As discussed herein, in various embodiments the endoscope 200 can be a flexible medical imaging instrument configured to allow the user to navigate anatomical passages or cavities that would otherwise be inaccessible by direct line of sight. The neck 210 of the endoscope 200 can include an elongated, flexible shaft extending distally from the endoscope body 220. The neck 210 can include one or more internal lumens or channels that house control cables, optical fibers, electrical wiring, illumination conduits, suction or irrigation channels, or combinations thereof. The flexibility of the neck 210 in various examples can allow the neck 210 to bend and conform to the shape of an anatomical pathway while maintaining a defined external diameter suitable for insertion. The outer surface of the neck 210 may be formed from a polymeric or composite sheath, such as polyurethane, silicone, or PTFE, optionally reinforced with a helical or braided metal coil to balance flexibility with torsional rigidity. In an un-actuated state, the neck 210 may be substantially straight, as shown in other figures, while actuation can cause the distal portion of the neck 210 to assume a curved or curled configuration, as illustrated in the example of FIG. 8.

The distal head 212 of the endoscope 200, (which may also be referred to as the distal tip), can house imaging and illumination components that enable the user to visualize internal anatomy. In some embodiments, the head 212 includes one or more lenses or optical windows coupled to a fiber-optic bundle or digital image sensor such as a CMOS or CCD element. The head 212 may further incorporate one or more light-emitting elements, such as LED modules or optical fibers delivering light from an external source, configured to illuminate the field of view at the distal end. Additional ports may be included for suction, irrigation, biopsy, or the passage of surgical instruments.

Actuation of the distal end of the neck 210, and consequently the orientation of the head 212, can be achieved through one or more mechanical control systems integrated into the endoscope body 220. In typical embodiments, the endoscope body 220 includes one or more control knobs, levers, or triggers that tension and release control wires running along the length of the neck 210. These control wires (e.g., made of stainless steel or high-tensile alloys) can be anchored near the distal end of the neck 210 and connected proximally to the actuating mechanism in the handle. When the user rotates or deflects a control knob, tension is applied asymmetrically to one or more of these wires, causing the distal portion of the neck 210 to bend in a controlled direction (e.g., up, down, left, right, or any intermediate angle). By coordinating multiple control inputs, the distal end can curl, as shown in FIG. 8, or return to a linear orientation as shown in other figures such as FIG. 3.

Visualization of the operative field is provided to the user via an imaging pathway that transmits the captured image from the head 212 to a display device. In fiber-optic endoscopes, this pathway can include a coherent fiber bundle transmitting the optical image directly to an eyepiece or an attached camera. In various examples of digital endoscopes, an image sensor within the head 212 converts optical information into electronic signals transmitted through internal wiring to a control console or video processor. The processed image can then be displayed in real-time on a monitor or integrated screen, allowing the operator to navigate and manipulate the endoscope while observing live feedback from the distal field of view.

In some embodiments, the endoscope 200 can include additional control systems that coordinate distal bending, axial translation, and rotation of the neck 210 to achieve precise orientation of the head 212 relative to a target site. These mechanisms can be manual, as described herein, or can incorporate automated components such as servomotors, linear actuators, or shape-memory alloys to control bending in response to user commands through a joystick, foot pedal, or software interface. In various examples, the ability to curl and un-curl the distal portion of the neck 210 as shown in FIG. 8 can provide the operator with fine control of the viewing direction and can enable the endoscope to access and visualize otherwise difficult-to-reach anatomical structures.

The examples herein should not be construed as limiting and the endoscope system discussed herein can be used in any suitable way for in human and/or animal inspection including in the nasal cavity (rhinoscopy), throat (laryngoscopy), bronchial tubes (bronchoscopy), esophagus (esophagoscopy), stomach (gastroscopy), small intestine (enteroscopy), colon (colonoscopy), bladder (cystoscopy), urethra (urethroscopy), joints (arthroscopy), abdominal cavity (laparoscopy), uterus (hysteroscopy), bile ducts (cholangioscopy), pancreatic ducts (pancreatoscopy), ear (otoscopy), sinuses (sinuscopy), thoracic cavity (thoracoscopy), spinal canal (epiduroscopy), fallopian tubes (falloposcopy), small blood vessels (angioscopy), and the like. Additionally, in some embodiments, endoscopes can be used to inspect non-living objects such as a building, vehicle, or the like.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives. Additionally, elements of a given embodiment should not be construed to be applicable to only that example embodiment and therefore elements of one example embodiment can be applicable to other embodiments. Additionally, in some embodiments, elements that are specifically shown in some embodiments can be explicitly absent from further embodiments. Accordingly, the recitation of an element being present in one example should be construed to support some embodiments where such an element is explicitly absent.

What is claimed is:

1. A method of using an endoscope, the method comprising:
coupling an endoscope adaptor system to an endoscope body of an endoscope, the endoscope adaptor system including:
a C-shaped coupling body has a front end that extends to a pair of rear ends that define a gap that defines an opening into a coupling cavity defined by the coupling body, the coupling body defining planar top and bottom faces,
an elongated linear rod that is coupled to and extends from the front end of the coupling body, from a rod base to a rod end, along an axis that is parallel to the planar top and bottom faces, the elongated linear rod comprising at least a first snap and a second snap, the first snap coupled to and extending from the elongated linear rod centrally between the rod base and the rod end, the second snap coupled to and extending from the elongated linear rod proximate to the rod end,
wherein the coupling the endoscope adaptor system to the endoscope body of the endoscope includes inserting a portion of the endoscope body into the coupling cavity via the gap that defines the opening into a coupling cavity,
wherein inserting the portion of the endoscope body into the coupling cavity causes the coupling body to flex and causes the gap to widen to allow the portion of the endoscope body to enter and be coupled within the coupling cavity;
coupling an elongated flexible neck of the endoscope to the rod by inserting the elongated flexible neck of the endoscope into the first snap and into the second snap, where a distal end of the elongated flexible neck includes a head that extends past the rod end, where the flexible neck of the endoscope is slidably coupled within the first snap and the second snap to allow the flexible neck of the endoscope to move along the length of the rod and to extend and retract the head of the flexible neck a desired length past the rod end;
holding, by a user, the endoscope body with a single hand of the user; and
performing an endoscopy task with the endoscope that includes:
inserting at least the head of the endoscope into the body of a subject,
manipulating, by the single hand of the user, the flexible neck of the endoscope to move the flexible neck along the length of the rod and to extend and retract the head of the flexible neck a desired length past the rod end to perform the endoscopy task while the user is holding the endoscope body with the single hand of the user, and
using fingers of the single hand to actuate a button or trigger of the endoscope to change the direction of the head of the endoscope while the user is holding the endoscope body with the single hand of the user.

2. The method of claim 1, wherein the first snap and the second snap are C-shaped or O-shaped.

3. The method of claim 1, wherein rod has a flat rectangular shape.

4. The method of claim 1, wherein the coupling body comprises four posts that line exterior faces of the rear ends of the coupling body, where the four posts serve as attachment points for one or more flexible straps or bands, are tethered across the gap and around the endoscope body to provide additional coupling support for the coupling body on the portion of the endoscope body.

5. A method of using an endoscope, the method comprising:
coupling an endoscope adaptor system to an endoscope body of an endoscope, the endoscope adaptor system including:
a coupling body has a front end that extends to a pair of rear ends that define a gap that defines an opening into a coupling cavity defined by the coupling body,
an elongated linear rod that is coupled to and extends from the front end of the coupling body, from a rod base to a rod end, the elongated linear rod comprising at least a first snap and a second snap, the first snap coupled to and extending from the elongated linear rod between the rod base and the rod end, the second snap coupled to and extending from the elongated linear rod proximate to the rod end;
coupling an elongated flexible neck of the endoscope to the rod by inserting the elongated flexible neck of the endoscope into the first snap and into the second snap, where a distal end of the elongated flexible neck includes a head that extends past the rod end, where the flexible neck of the endoscope is slidably coupled within the first snap and the second snap to allow the flexible neck of the endoscope to move along the length of the rod and to extend and retract the head of the flexible neck a desired length past the rod end; and
performing an endoscopy task with the endoscope that includes:
inserting at least the head of the endoscope into the body of a subject, and
manipulating, by a single hand of a user, the flexible neck of the endoscope to move the flexible neck along the length of the rod and to extend and retract the head of the flexible neck a desired length past the rod end to perform the endoscopy task while the user is holding the endoscope body with the single hand of the user.

6. The method of claim 5, wherein the coupling body is C-shaped.

7. The method of claim 5, wherein the coupling body defines planar top and bottom faces.

8. The method of claim 5, wherein the coupling the endoscope adaptor system to the endoscope body of the endoscope includes inserting a portion of the endoscope body into the coupling cavity via the gap that defines the opening into a coupling cavity, and
wherein inserting the portion of the endoscope body into the coupling cavity causes the coupling body to flex and causes the gap to widen to allow the portion of the endoscope body to enter and be coupled within the coupling cavity.

9. The method of claim 5, wherein performing the endoscopy task with the endoscope includes:
using fingers of the single hand to actuate a button or trigger of the endoscope to change the direction of the head of the endoscope while the user is holding the endoscope body with the single hand of the user.

10. A method of using an endoscope, the method comprising:
performing an endoscopy task with an endoscope that includes:
a rod that is coupled to the endoscope and that extends from a rod base to a rod end,
wherein performing the endoscopy task with the endoscope includes manipulating, by a hand of a user, a neck of the endoscope to move the neck along the length of the rod and to extend and retract a head of the neck a desired length past the rod end to perform the endoscopy task while the user is holding the endoscope.

11. The method of claim 10, further comprising inserting at least the head of the endoscope into the body of a subject.

12. The method of claim 10, further comprising coupling an endoscope adaptor system to an endoscope body of the endoscope.

13. The method of claim 12, wherein the endoscope adaptor system includes a coupling body has a front end that extends to a pair of rear ends that define a gap that defines an opening into a coupling cavity defined by the coupling body.

14. The method of claim 12, wherein the endoscope adaptor system includes the rod that is coupled to and extends from a front end of a coupling body of the endoscope adaptor system.

15. The method of claim 14, wherein the rod comprises a snap coupled to and extending from the rod proximate to the rod end.

16. The method of claim 15, further comprising coupling the neck of the endoscope to the rod by coupling the neck to snap.

17. The method of claim 15, wherein the neck of the endoscope is slidably coupled with the rod to allow the neck to slidably move along the length of the rod and to extend and retract the head of the neck a desired length past the rod end.

18. The method of claim 10, further comprising manipulating, by a single hand of a user, the neck of the endoscope to move the neck along the length of the rod and to extend and retract the head of the neck a desired length past the rod end to perform the endoscopy task while the user is holding an endoscope body of the endoscope with the single hand of the user.
